(19) United States
(12) Patent Application Publication
(10) Pub. No.: US 2010/0298643 A1
(43) Pub. Date: Nov. 25, 2010

(54) Endoscope With Eye-Piece Tiltable Via Metal Bellows

(76) Inventor: Hans-Peter Eisele, Tuttlingen (DE)

Correspondence Address: ST. ONGE STEWARD JOHNSTON & REENS, LLC, 986 BEDFORD STREET, STAMFORD, CT, 06905-5619, US

(21) Appl. No.: 12/784,265

(22) Filed: May 20, 2010

(30) Foreign Application Priority Data
May 20, 2009 (DE) 10 2009 022 118.2

(51) Int. Cl. A61B 1/06

(52) U.S. Cl. 600/162

(57) ABSTRACT

An endoscope, in particular for intubating an airway, includes a shaft, which in its distal end area includes a lens system for receiving an endoscopic image, and an endoscope head, which includes a first head section, which is connected with the shaft, as well as a second head section which includes an eyepiece, such that the second head section is configured tiltably in relation to the first head section, and such that the endoscope includes an image conductor, which is flexible at least in sections, for transmitting to the eyepiece the image captured by the lens system. The image conductor is surrounded by a protective tube that continues from the shaft as far as the second head section and is flexibly tiltable at least in sections. As a result, protection is afforded from both mechanical damage and from penetration of steam in superheated-steam sterilization.

### CROSS-REFERENCE TO RELATED APPLICATIONS

The present application claims priority of German patent application No. 10 2009 022 118.2 filed on May 20, 2009.

### FIELD OF THE INVENTION

The present invention relates to an endoscope, in particular an endoscope for intubating an airway.

### BACKGROUND OF THE INVENTION

Endoscopes of this type are used in particular in anesthesiology or emergency medicine to intubate a patient's airway. In the process, an intubation tube is inserted through the air pipes to supply the patient with air or oxygen. In particular with patients with anatomically determined intubation difficulty, it is necessary to control the correct insertion of the intubation tube. For this purpose the shaft of the endoscope is inserted through the patient's nose or mouth. To ensure that the physician can maintain an optimal position as the insertion of the endoscope shaft proceeds, a tiltable eyepiece section is provided. To transmit the endoscopic image from the distal end (that is, the end turned away from the physician) to the proximal end (toward the physician) of the endoscope, generic endoscopes make use of a flexible or semi-flexible image conductor, which consists as a rule of an optical fiber bundle.

U.S. Pat. No. 4,838,245 discloses an endoscope of this kind, which comprises a long shaft that can be tilted appropriately for insertion into a body cavity. Connected with the shaft is a head section to which an eyepiece section is joined in tiltable manner by a joint or hinge. A flexible image conductor, which serves to transmit the image from the distal end of the shaft to the eyepiece, is guided through the joint and allows a curvature of the eyepiece section. In this manner the physician, in inserting the endoscope into the patient's body, can easily follow the varying orientation of the endoscope shaft. Rotation of the endoscope around the longitudinal axis is transmitted by the joint without hindrance.

U.S. Pat. No. 5,377,668 discloses a fiberoptic endoscope, or fiberscope, which comprises one or more pivotable eyepiece members. The image conductor can be inserted into a disposable shaft, in which it is positioned movably to assume varying viewing angles.

According to patent EP 1,859,728 A2, an endoscope comprises a shaft and an eyepiece, such that the eyepiece is positioned on the proximal end of an endoscope head. A joint is provided between the shaft and the eyepiece to tilt the eyepiece relative to a longitudinal direction of the shaft. Thus the joint is configured in such a way that the eyepiece can be tilted in two planes. For this purpose the joint can, in particular, take the form of a ball joint or a universal joint. The image conductor in the area of the joint can be enclosed in a protective sleeve, which protects the image conductor from damage by the joint.

Patent DE 103 51 185 A1 discloses a superheated-steam-sterilizable endoscope with a housing, which is configured in its distal end region as a straight tubular shaft and in its proximal end region comprises a tiltable section with a rigid proximal endpiece connected to it. The housing is traversed by an image conductor, which is enclosed in the tubular shaft by a rigid protective tube that is closed before the lens system with a window. The protective tube ends at the distal end of the tilting section. In the area of the tilting section, the image conductor is surrounded by a flexibly tiltable metal bellows, which is affixed proximally on the endpiece and distally on the proximal end of the protective tube, insulated from steam. The housing itself is not steam-insulated.

The image conductor, which serves in the aforementioned endoscopes to transmit the endoscopic image, consists as a rule of an optical fiber bundle, which must be protected both from mechanical damage and from heated steam. Thus it is particularly essential in the area of the curvature to protect the image conductor from damage from friction during tilting, for instance through friction with the inside of a metal bellows surrounding the image conductor, such that the image conductor in particular can be damaged by friction from the corrugated structure of the metal bellows. Likewise, image conductors as a rule are sensitive to hot steam, which penetrates during superheated steam sterilization and, for instance, can lead to glass corrosion. In addition, moisture penetrating into an optical system can lead to coating of optical elements and thereby can disturb the functioning. Therefore protective action is necessary to prevent the penetration of hot steam, even under pressure conditions applying in superheated steam sterilization, especially in the area of the curvature.

It is the object of the present invention to provide an endoscope of the aforementioned type, which includes improved protection from the cited harmful effects.

### SUMMARY OF THE INVENTION

This object is achieved according to the invention by an endoscope with a shaft, which in its distal end area comprises a lens system for receiving an endoscopic image; an endoscope head, which includes a first head section, which is connected with the shaft, as well as a second head section, which comprises an eyepiece, such that the second head section is tiltably connected with the first head section; an image conductor, which is flexible at least in sections, for transmitting the image recorded by the lens system to the eyepiece; and wherein the image conductor is enclosed by protective tube that continues from the shaft as far as the second head section, and is flexibly tiltable at least in sections.

An inventive endoscope comprises a shaft that is configured for insertion into a cavity, in particular an internal cavity of a human or animal body. The shaft can be of rigid, flexible, or semi-flexible configuration. If the shaft is rigid or semi-flexible, then the shaft can be straight or else, at least in segments, tilted. Positioned in the interior of the shaft is a lens system that serves to acquire an image of an area that is to be observed, for instance a section of an airway. For transmitting the image acquired by the lens system, there is configured on the shaft an image conductor that consists of an optical fiber bundle, in particular an ordered optical fiber bundle.

In addition, an inventive endoscope includes an endoscope head, comprising a first and a second head section. The first head section is connected with the shaft. Thus the first head section can comprise a longitudinal axis that can correspond in particular with the longitudinal axis of the shaft. If the shaft is configured as tilting in sections, then the longitudinal axis of the first head section can correspond in particular with the longitudinal axis of the proximal end area of the shaft.

The second head section includes an eyepiece for observing the image transmitted by the image conductor by an observer, in particular by the physician. The second head section is connected with the first head section and can be tilted with respect to the first head section. Thus it becomes possible for the physician, independently of the orientation of the endoscope, always to assume an optimal physical posture. This is particularly important when the endoscope must be moved along a tilting path upon insertion into a body cavity, for instance because the body cavity itself tilts.

The image conductor continues from the shaft through the first head section into the second head section. For this purpose the image conductor, at least in the area of the connection between the first and second head sections, is sufficiently flexible in configuration to be able to follow the curvature of the second head section even with repeated movement.

According to the invention, the image conductor is surrounded by a protective tube that is flexibly tiltable, at least in the area of the connection between the first and second head sections, and that is configured continuously from the shaft all the way to the second head section. Because the protective tube is of flexibly tiltable configuration, curvature of the second head section becomes possible with respect to the first head section. A continuous protective tube offers the best possible protection against mechanical damage through friction with other components that can come in contact with the image conductor, particularly in the area of the connection between the first and second head sections. Finally, because the image conductor is encased in a continuous protective tube, in particular in a steam-tight continuous protective tube, a steam-tight protection of the image conductor becomes possible with respect to the environment. In particular, the image conductor as a result is protected against penetration of superheated steam upon sterilization in the area of the connection between the first and second head sections, without any additional insulating measures being required in this area.

In this case the protective tube is configured as flexibly tiltable in particular, but resistant to any pressure forces. It thereby becomes possible to avoid both any deformation of the protective tube from the impact of pressure forces in sterilization, and also any damage to the image conductor upon tilting the protective tube or through contact with other components upon tilting the second head section.

Possible materials for the protective tube include, in particular, sufficiently deformable metallic materials, for instance NiTi alloys (nitinol). Such metallic protective tubes offer excellent insulation against hot steam as well as sufficient resistance to lateral pressures. If somewhat lower requirements are made in terms of insulation from hot steam, protective tubes of certain synthetics can be used if they have sufficient thermal and mechanical stability and are admissible for use in endoscopes, for instance polyetheretherketone (PEEK). It is also possible to use multi-layered protective tubes, which for instance comprise various layers to ensure insulation against steam and also mechanical stability.

The protective tube in the area of the connection between the first and second head sections is preferably sized so that during tilting the image conductor makes no contact, or only limited contact, with the inner wall of the protective tube. Damage from friction is thus avoided, even with multiple tilting movements as well as in further handling of the endoscope. In addition the interior wall of the protective tube is preferably configured in such a way that, even with possible contact with the image conductor, the friction remains as limited as possible. For this purpose, the interior wall can be configured in particular as smooth, that is, without a corrugated surface or with the least possible abrasiveness, or coated with a friction-reducing layer, for instance polytetrafluorethylene (PTFE). Likewise, the image conductor inside the protective tube can also be encased in a friction-reducing protective sleeve, possibly of PTFE.

According to a preferred embodiment, the protective tube encloses the lens system at least partially. In this case, the protective tube continues to the proximity of the distal end of the shaft and extends beyond the distal end of the image conductor. The lens system, which as a rule consists of an arrangement of several lenses, is enclosed by the part of the protective tube that extends beyond the distal end of the image conductor. This improves the mechanical stability of the arrangement of the lens system and image conductor.

The protective tube is preferably connected in steam-tight manner with the periphery of a lens, in particular of the distal-end lens of the lens system, or with the peripheral surface of a frame or sleeve, which encases an individual lens or the lens system as a whole. Consequently the connection of the protective tube on the distal end is made steam-tight, so that the image conductor and lens system are protected from steam penetration.

Likewise, the protective tube can enclose the eyepiece, which as a rule is constructed of several lenses. In this case the protective tube extends beyond the proximal end of the image conductor. The eyepiece, which as a rule consists of an arrangement of several lenses, is enclosed by the part of the protective tube that extends beyond the proximal end of the image conductor. This results in an improved mechanical stability of the arrangement of the eyepiece and the image conductor.

The protective tube is preferably connected in steam-tight manner with the periphery of an eyepiece lens, in particular with the proximal-end lens of the eyepiece, or with the outside surface of a frame or sleeve, which encloses an individual lens or the eyepiece as a whole. As a result, a steam-tight connection of the protective tube becomes possible on the proximal end, so that the image conductor and eyepiece are protected from steam penetration.

If both the distal and the proximal ends of the protective tube are insulated against steam in this manner, then the protective tube constitutes a steam-tight unit through which the image conductor as well as the lens system and eyepiece are especially securely protected from steam penetration.

According to another preferred embodiment, the protective tube is configured as a single unit. In this case the protective tube comprises no connecting sections in which the danger of penetration by hot steam could exist. In this manner, a particularly strong security against the penetration of hot steam is achieved, even with multiple sterilizations and multiple curvature of the second head section. In addition, the structure is particularly simple and allows cost savings in production with a reduced number of manufacturing stages.

Alternatively to the foregoing, the protective tube can be configured in multiple sections. Thus the protective tube can, for instance, consist of a different material in the shaft area than in the area of the connection between the first and second head sections. Consequently, through appropriate selection of diameter, wall strength, and material, it is possible to achieve optimal properties for the respective area. Thus, for instance, in the area of the shaft a high degree of mechanical stability can be desirable, along with the requirement of a small diameter as demanded by the application, while in the area of the connection between the first and second head sections sufficient deformability is called for, also at greater diameter, to realize the curvature.

Preferably the protective tube should be configured in multiple parts in such a way that in the area of the shaft it consists of a metallic material with high rigidity and good possibility of combination with a paired component by welding, soldering, cementing, or shrinking. Even at small wall thickness, this makes it possible to stabilize the shaft and image conductor. The protective tube can be in close proximity adjacent with the image conductor in this area. In the area of the connection between the first and second head sections, the protective tube on the other hand has a greater diameter, so that the image conductor is as little as possible in contact with the interior wall of the protective tube; the protective tube in this area is made of a material that ensures sufficient flexibility. Thus the various parts of the protective tube are connected with one another as steam-tight as possible by cementing, welding, or soldering.

In another preferred embodiment of the invention, the protective tube in the area of the connection between the first and second head sections is enclosed by a metal bellows. In this area a tilting of the image conductor and thus a tilting of the protective tube must be possible in order to permit the curvature of the second head sections. Because a flexibly tiltable metal bellows is positioned in the section of the protective tube in which the curvature occurs, the protective tube and thus in addition the image conductor are protected from mechanical damage in the curvature. In addition, the metal bellows can prevent creasing of the protective tube during strong curvature or even from very frequent curvature. For this purpose the metal bellows can constitute a forced turning of the protective tube. To reduce friction, between the metal bellows and the protective tube it is possible to provide, for instance, a sleeve made of PTFE.

According to a particularly preferred embodiment of the invention, the metal bellows in its distal end area is connected in steam-tight manner with a housing of the first head section. In particular, the first head section is provided with a steam-tight housing, which can be connected in steam-tight manner with the shaft or insulated from the shaft. To achieve a steam-tight connection between the metal bellows and the housing, a cylindrical distal end section of the metal bellows can be inserted in a corresponding borehole of the housing and connected in insulated manner with the housing, for instance through cementing, welding, or soldering.

Thereby, additional security can be provided against penetration of hot steam during sterilization. In this manner, the internal area of the housing is already configured to a great extent in steam-tight manner, so that the requirements in terms of insulation of the protective tube can be lessened. In this case in particular, a protective tube can also be used, consisting at least in sections of a synthetic material such as PEEK and comprising a lower degree of steam insulation with respect to metallic materials. The transition between the two parts of the protective tube can be positioned, for instance, inside the first head section. As a result, a particularly flexible configuration of the protective tube becomes possible in the area of the curvature.

The proximal end section of the metal bellows can be connected with the protective tube in insulated manner by cementing, welding, or soldering. In preferred manner, in its proximal end area the metal bellows is connected in steam-tight manner, however, with a housing of the second head section. Said housing is likewise configured as insulated against steam. This provision has the advantage that a high degree of steam insulation already exists within the housing of the second head section, so that not only is it possible to reduce the requirements in terms of steam insulation of the protective tube in this area, but also the optical elements positioned in the second head section, in particular the eyepiece lenses and proximal window, are protected from penetration of moisture.

According to another preferred embodiment, the second head section is connected with the first head section by a joint, which allows a curvature of the second head section in one plane. Consequently a controlled curvature becomes possible. This has the additional advantage that after a curvature the second head section can be securely returned to the starting position without leaving in place a multiple, snake-shaped curvature of a flexibly tiltable curvature section or, resulting from this, a displacement of the second head section. This provision therefore contributes toward controlled handling as well as increased durability. In addition, a joint allows for secure transmission of torque if a rotation of the endoscope is required around the longitudinal axis, for instance to insert a tilted point of the endoscope shaft into a body cavity to match the anatomical particularities or, in the case of a lateral viewing lens, to select the viewing angle according to the requirements.

In another embodiment of the invention, the joint can allow a curvature of the head section in several planes. Consequently a particularly flexible handling becomes possible.

According to another preferred embodiment of the invention, the possible range of the angle of curvature is restricted. Thus, for instance with a curvature in one plane, the possible angle range can be restricted to 45 degrees or preferably 30 degrees, calculated from a straight position of the second head section relative to the first head section. This has the advantage that extreme curvatures, which could lead to damaging of the image conductor or protective tube, are securely avoided. For a curvature in several planes, such a restriction can exist in each plane or also in any possible space alignment, calculated from the longitudinal axis of the first head section.

In a particularly preferred embodiment, the angle range of the curvature is restricted asymmetrically to the longitudinal axis of the first head section. Because in using the inventive endoscope a curvature in one direction is preferred, it becomes possible as a result to reduce the admissible angle range without essential restriction of applications, and consequently to reduce the danger of damage, for instance through exhaustion.

In particular, for the expected application of the endoscope, it is sufficient as a rule that a curvature of the second head section from a straight position is possible only in one direction. Therefore, calculated from the longitudinal axis of the first head section, the second head section, or a longitudinal axis associated with it, can have an angle range, for instance, of zero to 45 degrees or preferably zero to 30 degrees. Such a reduced angle range allows an additional extension of the lifetime of the endoscope without essentially restricting its application.

According to another preferred embodiment of the invention, a friction-inducing element is provided in the joint. As a result of this frictional contact, it can become possible for the second head section to securely retain a curvature once it has been selected by the physician, withstanding any possible return force of the image conductor or protective tube and independently of any possible impact of handling forces or of gravitational force. The angle position can be modified only by exerting a force greater than that of manual strength.

In preferred manner, the friction is adjustable. This has the advantage that the desired friction can be selected specifically for a desired application among a range of possibilities. Adjustability of the friction can also be advantageous for compensating for diverse or variable material properties. The selection of the friction can occur exclusively at the manufacturing stage or else can be possible during application by the user.

The joint, in particular, can be configured in such a way that an axis can be connected with one of the head sections that can tilt with respect to one another, whereby said axis engages in a joint disc that is connected with the other head section. If, for instance, an axis that is oriented perpendicular to the longitudinal axis of the first head section is connected with the first head section, then the second head section can comprise a joint disc into which the axis engages. Here the axis can also be configured in particular as interrupted or in multiple parts to make possible unimpeded action of the image conductor with the surrounding protective tube. For this purpose the joint disc can comprise a borehole. Said borehole can be executed in such a way that the joint disc avoids contact with the protective tube or with a metal bellows surrounding said tube during admissible curvature. In preferred manner the borehole is positioned eccentrically. This provision has the advantage that more space is available for receiving additional elements, for instance springs to produce or select the friction. In addition, the eccentric positioning of the borehole makes possible an eccentric arrangement of the protective tube or of the metal bellows, so that at a unilateral curvature area it is possible to minimize friction between the image conductor and the protective tube.

The inventive endoscope is especially suited for use in intubating an airway. The range of application of the invention, however, is not restricted to this application, but rather the advantages of the invention can be used in a number of other endoscopic applications.

It is understood that the properties mentioned so far as well as those to be described hereinafter can be used not just in the particular indicated combination but also in other combinations or alone, without departing from the context of the present invention.

Further aspects of the invention are discussed in the following description of a preferred embodiment and the appended illustrations.

### BRIEF DESCRIPTION OF THE DRAWINGS

FIG. 1 presents a general view of an inventive endoscope.

FIG. 2 shows the endoscope head of an inventive endoscope, including a straight and a curved position of the second head section.

FIG. 3 shows a partly cut-out depiction of the endoscope head of an inventive endoscope.

FIG. 4 shows a perspective, partly opened view of the joint of an inventive endoscope.

FIG. 5 shows an enlarged longitudinal section through the first head section of an inventive endoscope.

FIG. 6 shows a partly cut-out view of an endoscope head of another embodiment of the invention.

### DETAILED DESCRIPTION OF THE INVENTION

According to FIG. 1, an inventive endoscope 1, which can also be designated as an endoscope lens system, comprises a long, straight shaft 2 and an endoscope head 3. The shaft is configured for insertion into a body cavity and includes a distal end area 4, which can be configured as straight, a tilted section 5, and a proximal section 6. A number of markings 7 can be applied on the shaft that give the physician an indication of how far the shaft has already been inserted into the body cavity.

The endoscope head 3 comprises a first head section 10 and a second head section 20. The first head section 10 is connected by a cone 11 with the proximal section 6 of the shaft 2. Seated on the first head section 10 is a light connection 12, to which a light-conducting cable can be attached for introducing an illuminating light that is generated in an eternal light source and is conducted to the distal end 8 of the endoscope and exits there. It is also possible to provide an internal light source, such as in the endoscope head 3 or in the distal end area 4 of the shaft. The second head section 20 is connected with the first head section 10 by a joint 30. The second head section includes an eyepiece cup 21 for direct viewing by an observer, in particular by a physician. The eyepiece cup can also be configured for connecting a video camera, for instance for documentary or training purposes. Gripping troughs 22 for improved handling can be positioned on the outside of the first and second head sections.

The distal end section 4 of the endoscope 1 contains a lens system (not illustrated) for receiving an endoscopic image of an image field, which is located in front of, diagonally to or laterally to the distal end area 4 of the endoscope, depending on the configuration of the lens system. The image taken by the lens system is conducted by an image conductor (not shown in FIG. 1) in the shaft 2 through the first head section 10 and through the joint 30 to the second head section, where the image can be observed through the eyepiece cup 21 by an eyepiece (not shown) that is incorporated in the second head section 20. In each case, a window (not shown) can be provided as distal light inlet or as proximal light outlet. In addition, the endoscope 1 can comprise a working channel for inserting instruments as well as channels and corresponding connections for gases and liquids (not shown).

As shown in FIG. 2, the second head section 20 can be pivoted from a straight position 27 into a tilted position 27′. In preferred manner this occurs in a plane, which is formed by the proximal section 6, the tilted section 5, and the distal end section 4 of the shaft as well as the light connection 12, in the direction toward the light connection 12. In the process the joint 30 preferably comprises such a degree of friction that the second head section remains in the tilted position 27′ without additional force being imposed. The second head section likewise remains in the straight position 27 as well as in every other position.

FIG. 3 shows the joint 30 and the immediately adjoining areas of the first and second head sections in a partly cut-out view. The first head section 10 comprises a housing 13, which is pierced by a cylindrical borehole 14 in the direction toward the joint 30. The second head section includes a housing 23, which comprises a cylindrical borehole 24 in the direction toward the joint 30. The first head section 10 can be of cylindrical configuration at least in sections, and comprises a longitudinal axis 15, which essentially corresponds with the longitudinal axis 9 of the proximal shaft section 6. The second head section 20 can likewise be configured cylindrically, at least in sections. In the straight position shown in FIG. 3, the longitudinal axis 25 of the second head section essentially corresponds with the longitudinal axis 15 of the first head section. The borehole 14 runs parallel, but eccentrically, that is not coaxially, to the longitudinal axis 15 of the first head section 10, just as the borehole 24 runs eccentrically to the longitudinal axis 25 of the second head section 20.

The joint comprises a joint disk 31, which in the embodiment is connected with the second head section 20 and is mounted so that it can rotate with respect to the first head section 10 with reference to a rotation point 33. The joint disc 31 is pierced by a borehole 32 that is eccentric with respect to the longitudinal axis 25 of the second head section and through which a flexibly tiltable metal bellows 34 is conducted, which has a corrugated structure. The metal bellows 34 includes a distal end area 35 and a proximal end area 36. The distal end area 35 of the metal bellows 34 is inserted into the borehole 14 and receives a steam-tight connection there with the housing 13, just as the proximal end area 36 is inserted into the borehole 24 and receives a steam-tight connection there with the housing 23. Both the housing 13 of the first head section 10 and the housing 23 of the second head section 20 are of steam-insulated configuration. The protective tube and image conductor are not shown in FIG. 3.

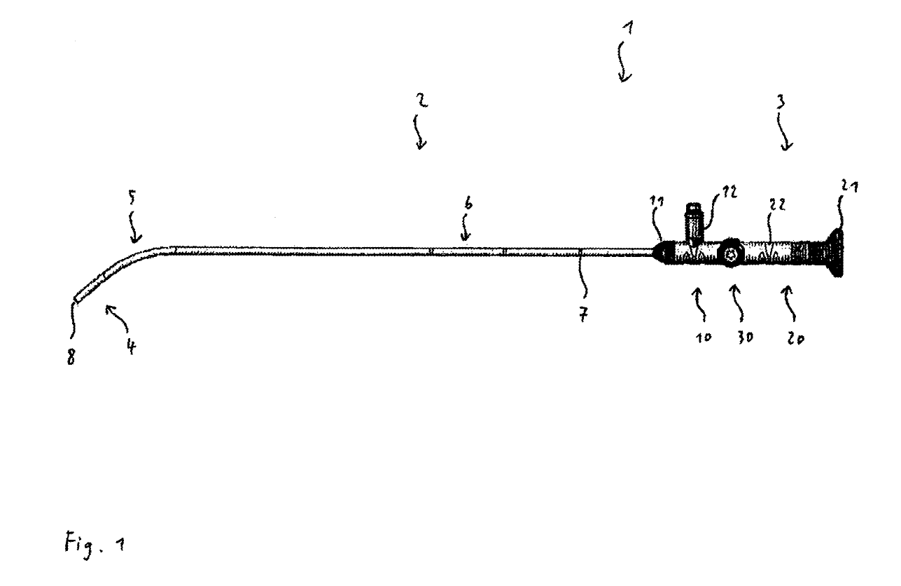

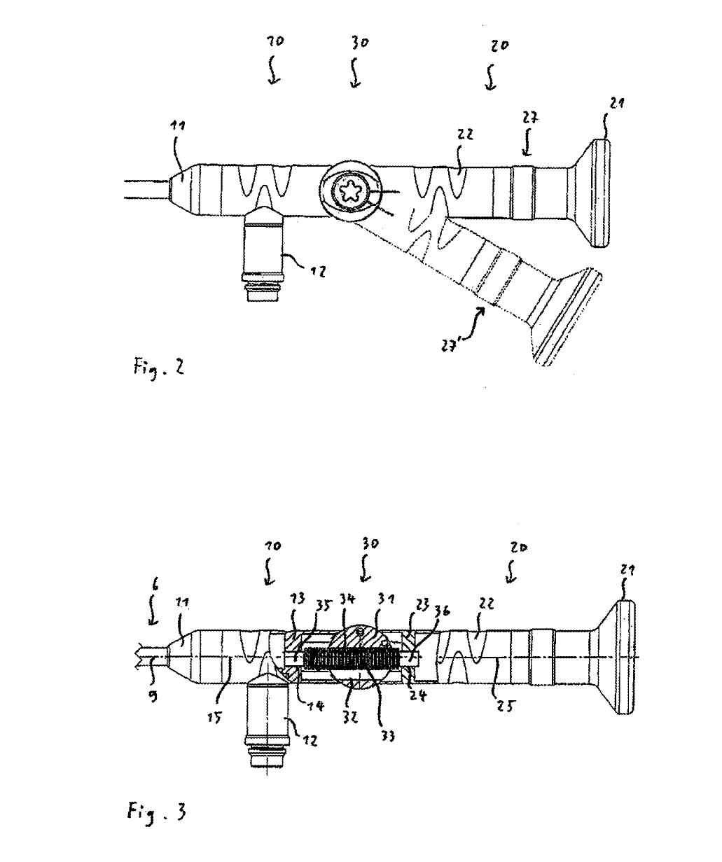

In FIG. 4 the joint 30 is partly opened and is shown in perspective view together with the adjoining areas of the first and second head sections. Two disks 38 are connected on both sides of the joint with the first head section 10 (only the rear disk is shown in FIG. 4) and comprise bevelings 39 for better handling. A pin 37 engages in a long hole of a disk 38 (not shown) to restrict the pivot area of the second head section. Insertion points 40 are configured as a single unit with the housing 13 of the first head section and comprise boreholes 41 in which screws (not illustrated) are inserted for securing the pivot axis 33′ that serve to secure the rotation axis 33′ and engage in the joint disc 31 and through which the friction caused by sliding discs 42 can be adjusted.

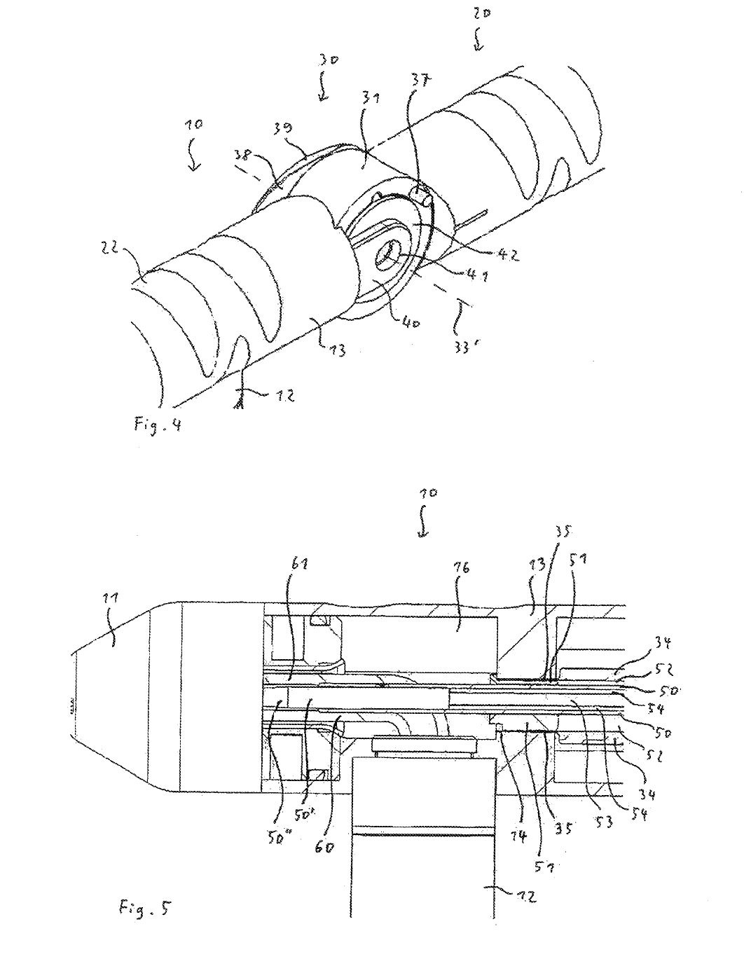

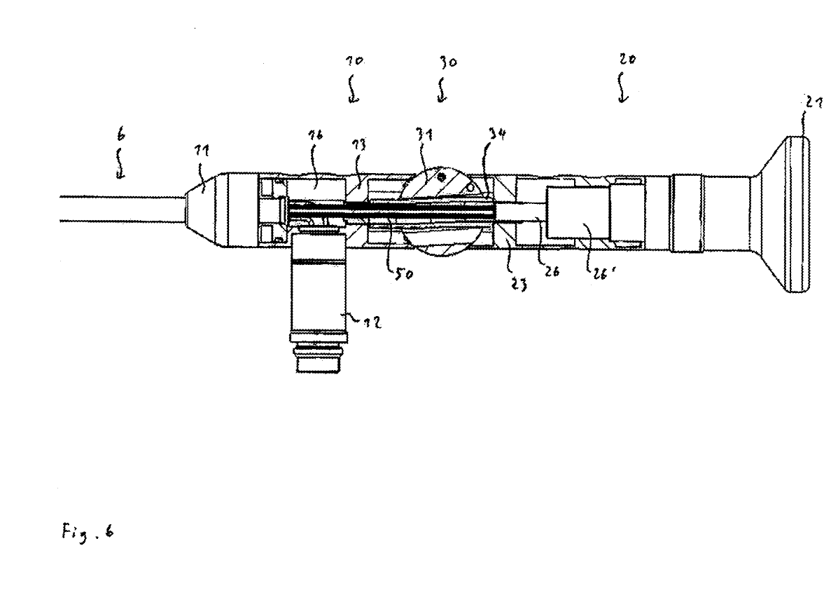

According to FIG. 5 the bellows 34 comprises a distal end section 35 that is inserted in the borehole 14 of the housing 13 and is connected in steam-tight manner with it, for instance by welding, cementing, soldering, or laser soldering. A section 50 of the protective tube is positioned eccentrically inside the metal bellows and held by holders 51. The intermediate space 52 between the metal burrows 34 and the protective tube 50 serves to allow the necessary movement of the protective tube relative to the metal bellows; to reduce friction, a tube made of PTFE for instance can be inserted in the intermediate space 52. The image conductor 53 consisting of an optical fiber bundle is positioned inside the protective tube 50 and essentially coaxially with the longitudinal axis 15 (not illustrated) of the first head section. The intermediate space 54 between the protective tube and the image conductor serves to prevent friction between the protective tube and image conductor and can also be partly supplemented by a means for reducing friction, for instance a tube of PTFE.

Also attached to the first head section 10 is the light connection 12, through which light conductors 60, 61 consisting of optical fiber bundles are conducted into the interior 16 of the housing 13. Light conductors 60, 61 are conducted through the shaft as far as the distal end 8 of the endoscope, where the illumination light coupled to the light connection 12 to illuminate an endoscopic image field emerges. The light connection is positioned in steam-tight manner on the housing 13. The housing 13, in addition, is sealed off in steam-tight manner from the shaft 2, so that the interior area 16 of the housing 15, together with the housing 23 connected to it by the metal bellows, is sealed off in steam-tight manner altogether.

Inside the interior space 16 the protective tube, which can consist for instance of PEEK in the section labeled as number 50, leads to a section 50′, which can consists for instance of a metallic substance and leads by means of a spherical section 50″ into a section of the protective tube that runs inside the shaft and likewise consists of a metallic substance. The sections 50′ and 50″ as well as the section (not illustrated) running inside the shaft can, in particular, form a single unit, while the section 50 of the protective tube is connected in steam-tight manner with the section 50′.

FIG. 6 shows in a partly cut-out view another embodiment of the invention, in which the protective tube 50 is configured altogether as a single unit. As can be seen in FIG. 6, the protective tube 50, coming from the shaft 6, is conducted through the first head section 10 and the joint 30 as far as the second head section 20 and inside this into a guide sleeve 26, 26′. Further details and reference numbers correspond to those of the aforementioned embodiment.

In an additional embodiment of the invention, not illustrated, the second head section can also comprise a tilted basic position from which the curvature occurs; the first head section in this case can also extend in the proximal direction beyond the connection area with the second head section and comprise, for instance in this area, an entry for an instrument channel in the proximal direction.

1. An endoscope, comprising:
a shaft, which in its distal end area comprises a lens system for receiving an endoscopic image;
an endoscope head, which includes a first head section, which is connected with the shaft, as well as a second head section, which comprises an eyepiece, such that the second head section is tiltably connected with the first head section, and
an image conductor, which is flexible at least in sections, for transmitting the image recorded by the lens system to the eye-piece; and
wherein the image conductor is enclosed by protective tube that continues from the shaft as far as the second head section, and is flexibly tiltable at least in sections.

2. The endoscope according to claim 1, wherein the protective tube at least partly encloses the lens system and/or the eyepiece.

3. The endoscope according claim 1, wherein the protective tube is configured as a single unit.

4. The endoscope according to claim 1, wherein the protective tube is configured in multiple parts.

5. The endoscope according to claim 1, wherein the protective tube is surrounded by a metal bellows in the area of a curvature section.

6. The endoscope according to claim 5, wherein the metal bellows is connected in steam-tight manner in its distal end area with a housing of the first head section.

7. The endoscope according to claim 6, wherein the metal bellows is connected in steam-tight manner in its proximal end area with a housing of the second head section.

8. The endoscope according to claim 1, wherein the second head section is connected with the first head section by a joint that allows a curvature of the second head section in at least one plane.

9. The endoscope according to claim 8, wherein the joint is configured in such a way that the angle area of curvature in the at least one plane is restricted.

10. The endoscope according to claim 9, wherein the angle area of the curvature is positioned asymmetrically to a longitudinal axis of the first head section, in particular that a curvature of the second head section from a straight position is possible only in one direction.

11. The endoscope according to claim 8, wherein the joint includes a friction-producing element.

12. The endoscope according to claim 11, wherein the friction is adjustable.

13. The endoscope according to claim 8, wherein the joint includes a joint disc, which comprises a borehole for conducting the protective tube surrounding the image conductor, such that the borehole is positioned eccentrically.

14. The endoscope according to claim 5, wherein the protective tube is positioned eccentrically in the metal bellows).
